(19) United States
(12) Patent Application Publication
(10) Pub. No.: US 2020/0146947 A1
(43) Pub. Date: May 14, 2020

(54) LEAVE-ON TYPE COSMETIC COMPOSITION FOR FACIAL MASK SHEET HAVING SELF-FOAMING FUNCTION

(71) Applicant: COSMAX, INC., Gyeonggi-do (KR)

(72) Inventors: Chan Gu KIM, Gyeonggi-do (KR); Ho-Yeol KIM, Gyeonggi-do (KR); Yoon Nam JO, Gyeonggi-do (KR); IL Rang LEE, Gyeonggi-do (KR); Ja Yoon KOO, Gyeonggi-Do (KR); Myeong Sam PARK, Seoul (KR)

(21) Appl. No.: 16/495,970

(22) PCT Filed: Mar. 19, 2018
(86) PCT No.: PCT/KR2018/003144

§ 371 Date: Sep. 20, 2019

(30) Foreign Application Priority Data
Mar. 21, 2017 (KR) 10-2017-0035254

(51) Int. Cl. A61K 8/02; A61K 8/60; A61K 8/34; A61K 8/81; A61K 8/70; A61Q 19/02; A61Q 19/08; A61Q 19/10

(52) U.S. Cl. CPC A61K 8/0212; A61K 8/602; A61K 8/345; A61K 8/8152; A61K 2800/30; A61Q 19/02; A61Q 19/08; A61Q 19/10; A61K 8/70

(57) ABSTRACT

The present invention relates to a cosmetic composition for a leave-on type mask sheet including a fluorinated compound and saponin as a self-foaming agent. The cosmetic composition for a leave-on type mask sheet includes a fluorinated compound and saponin as a self-foaming agent, and thus can provide a cleansing effect, such as an effect of removing sebum or waste. In addition, since the cosmetic composition includes saponin instead of a chemical surfactant, it can reduce side effects, such as skin irritation, alleviate inconvenience of washing-off after using the mask sheet, and improve a moisturizing effect by virtue of elimination of the washing-off procedure.

### FIELD

The present invention relates to a cosmetic composition for a leave-on type mask sheet having a self-foaming function.

### BACKGROUND

In recent society, personal hygiene has been given increasing attentions due to air pollution and environmental pollution caused by industrialization and an increase in traffic volume. Thus, women have given more attentions to cleansing makeup than wearing makeup. Therefore, various products related with cleansing have been launched in the market, and mask pack and cleansing products containing ingredients required for adsorption of contaminants and skin cleansing have been sold.

A surfactant is a material that is adsorbed to an interface and reduces free energy thereof, thereby causing a significant change in interfacial properties, and is an amphipathic material having both a hydrophilic group with affinity to water and an oleophilic group with affinity to oil in one molecule. Such surfactants are classified broadly into chemically synthesized chemical surfactants and naturally occurring natural surfactants. In addition, chemical surfactants include non-ionic surfactants, anionic surfactants, cationic surfactants, amphipathic surfactants, polymeric surfactants, or the like.

Particularly, a surfactant applied to cosmetics has been used as a main ingredient of cosmetics. In the cosmetics, the surfactant has a cleaning function (e.g. used for face cleansers, hair cleansers, body cleansers, etc.), emulsifying function (e.g. used for cream, lotion, etc.) and a solubilizing function (e.g. used for dissolving fragrance oil in a water-based product).

A surfactant that has been used frequently to date in the field of cosmetics is a chemical surfactant. However, such a chemical surfactant causes significant skin irritation, shows excessive defatting property to cause drying of the horny layer and makes the skin rough. In addition, the chemical surfactant has a side effect of causing inflammation and skin diseases. Thus, many studies have been conducted continuously in recent times to develop a natural surfactant capable of minimizing skin irritation.

To solve the problems of adsorption of contaminants and skin cleaning, commercially available mask sheet products have been based on self-foaming mask sheets (Korean Patent Laid-Open No. 10-2015-0108611) using a chemical surfactant and a volatile fluorinated ingredient, mask sheets in which carbonic acid gas generation is induced through the neutralization between the first agent and the second agent, or the like.

Since a chemical surfactant has been used in the mask sheets according to the related art, it may cause side effects of skin irritation, skin drying caused by skin defatting property and skin diseases, such as skin inflammation, when it remains on the skin for a long time. Therefore, after the mask sheets were used, they should be washed off inconveniently in order to overcome the above-mentioned side effects. Moreover, it is difficult to impart a skin moisturizing effect due to the washing-off procedure. Further, the mask sheets according to the related art have drawn attention merely in cleansing applications due to such essential washing-off procedure.

Under these circumstances, the inventors of the present invention have conducted many studies to overcome the above-mentioned problems. We have found that a cosmetic composition for a leave-on type mask sheet including a fluorinated compound and saponin as a self-foaming agent can reduce side effects, such as skin irritation, by virtue of the use of saponin instead of a chemical surfactant, alleviate inconvenience of washing-off after using the mask sheet, increase a moisturizing effect by virtue of elimination of the washing-off procedure, prevent loss of the functional ingredients contained in the cosmetic composition and wrinkle-improving and skin-whitening active ingredients of saponin during washing, and thus can provide higher wrinkle-improving and skin-whitening effects. In addition, we have found that the cosmetic composition also shows a cleansing effect of removing sebum and waste by using foam generated by self-foaming of saponin and a fluorinated compound. The present invention is based on these findings.

### REFERENCES

(Patent Document 1) KR10-2015-0108611 A

### DISCLOSURE

### Technical Problem

A technical problem to be solved by the present invention is to provide a cosmetic composition for a leave-on type mask sheet which includes saponin as a self-foaming agent instead of a chemical surfactant, and thus can reduce side effects, such as skin irritation, alleviate inconvenience of washing-off after using the mask sheet, increase a moisturizing effect by virtue of elimination of the washing-off procedure, and can provide a cleansing effect of removing sebum and waste.

Another technical problem to be solved by the present invention is to provide a cosmetic composition for a leave-on type mask sheet having a self-foaming function, which prevents loss of the functional ingredients contained in the cosmetic composition and wrinkle-improving and skin-whitening active ingredients of saponin during washing, and thus can provide higher wrinkle-improving and skin-whitening effects.

### Technical Solution

In one general aspect, there is provided a cosmetic composition for a leave-on type mask sheet which includes a fluorinated compound and saponin as a self-foaming agent.

The cosmetic composition for a mask sheet according to the present invention uses saponin, which has not been used as a surfactant in the field of cosmetics according to the related art, in combination with a fluorinated compound to allow formation of air bubbles of the self-foaming agent. In other words, air bubbles are formed spontaneously when the fluorinated compound is evaporated by the skin temperature and meets saponin having interfacial tension in the composition at the same time.

As used herein, the term ‘self-foaming’ means spontaneous formation of foam without physical force, such as rubbing or scrubbing. In addition, the term ‘self-foaming agent’ means a material which causes the above-mentioned self-foaming.

In the case of the self-foaming type mask sheet containing a fluorinated compound and a chemical surfactant according to the related art, skin sebum and waste were removed by using foam generated by the combination of the fluorinated compound with the chemical surfactant. However, in the mask sheet, since it includes a chemical surfactant in the cosmetic composition, it causes a skin irritation phenomenon due to the surfactant. Thus, after using the mask sheet, washing-off procedure should be performed. Moreover, due to the washing-off procedure, it was difficult to provide the skin with a sufficient moisturizing effect and the mask sheet was used merely for cleansing applications. However, when saponin is used instead of a chemical surfactant according to the present invention, foam is generated (self-foaming is accomplished) through the reaction of the fluorinated compound with saponin without any chemical surfactant and skin sebum and waste can be removed thereby. In addition, since no chemical surfactant is used, the washing-off procedure can be eliminated after using the mask sheet. Thus, it is possible to provide an improved moisturizing effect and convenience in use. Further, it is possible to prevent loss of the functional ingredients contained in the cosmetic composition and wrinkle-improving and skin-whitening active ingredients of saponin during washing, and thus to provide higher wrinkle-improving and skin-whitening effects. In addition, since the cosmetic composition for a leave-on type mask sheet according to the present invention avoids a need for washing-off procedure, it can be used for moisturizing, whitening and wrinkle-improving applications as well as for cleansing application. Moreover, in the case of the cosmetic composition for a leave-on type mask sheet according to the related art, it is merely a simple mask sheet containing functional ingredients, such as wrinkle-improving and whitening ingredients. However, the cosmetic composition for a mask sheet according to the present invention can also provide visual beauty and pleasure of foam generation through self-foaming.

As used herein, the term ‘leave-on type’ refers to allowing a cosmetic to be absorbed to the skin as it is without washing-off after using a mask sheet. According to the present invention, since no chemical surfactant causing skin irritation is used, there is no need for washing off a mask sheet after use and the functional ingredients contained in the cosmetic composition and wrinkle-improving and skin-whitening active ingredients of saponin are allowed to be absorbed to the skin as they are, thereby providing various effects.

In addition, the term ‘cosmetic composition for a mask sheet’ means a cosmetic composition with which a mask sheet made of a non-woven web, cellulose, hydrogel, etc. is impregnated. Preferably, the cosmetic composition according to the present invention is used for impregnating a mask sheet therewith.

According to the present invention, the cosmetic composition includes no chemical surfactant. Surfactants are classified broadly into chemically synthesized chemical surfactants and naturally occurring natural surfactants. A chemical surfactant is prepared artificially through chemical synthesis of two or more chemical ingredients. Chemical surfactants include non-ionic surfactants, anionic surfactants, cationic surfactants, amphipathic surfactants, polymeric surfactants, or the like.

Saponin used according to the present invention has not been used as a surfactant in the field of cosmetics, since it shows insufficient foaming capability or emulsifying ability, as compared to chemical surfactants. At first, the inventors of the present invention anticipated that saponin had insufficient foaming capability. However, it has been unexpectedly found that saponin reacts with a fluorinated compound and causes self-foaming sufficiently even without any chemical surfactant. In addition, saponin is advantageous in that it causes little skin irritation, unlike a chemical surfactant.

According to the present invention, the cosmetic composition is for combined use of cleansing, moisturizing, wrinkle-improving and whitening.

The cosmetic composition for a leave-on type mask sheet fundamentally shows a cleansing effect, since foam formed by the fluorinated compound and saponin removes skin sebum and waste. In addition, the cosmetic composition can be retained for a long time without washing-off with water, and thus it is possible to improve a moisturizing effect. Further, the wrinkle-improving and whitening functional ingredients (e.g. adenosine as a wrinkle-improving functional ingredient and niacin amide as a whitening functional ingredient) contained in the cosmetic composition and wrinkle-improving and skin-whitening active ingredients of saponin are allowed to be absorbed to the skin as they are, and thus it is possible to provide skin wrinkle-improving and whitening effects.

Particularly, according to the test examples of the present invention, the cosmetic composition (Example 1) for a leave-on type mask sheet according to the present invention was determined for the cleansing, moisturizing and skin-emollient effects in comparison with a cosmetic composition (Comparative Example 1) including a chemical surfactant for a cleansing type mask sheet. After the test, it was shown that both Example 1 and Comparative Example 1 show an excellent cleansing effect, such as a sebum-removing effect, but Example 1 shows higher moisturizing and emollient effects as compared to Comparative Example 1. In other words, Comparative Example 1 shows an excellent cleaning effect due to the use of a chemical surfactant but provides lower moisturizing and emollient effects due to the washing-off procedure after use. On the contrary, according to the present invention, there is no need for washing-off procedure after use, and thus it is possible to provide higher moisturizing and emollient effects as well as an excellent cleansing effect. As can be seen from the results, since the cosmetic composition according to the present invention uses saponin instead of a chemical surfactant, it not only shows excellent cleansing and moisturizing effects but also is safe to the skin. Therefore, it is possible to provide a cosmetic composition for a leave-on type mask sheet having various effects (see, Test Example 1).

In addition, the cosmetic composition according to the present invention causes little skin irritation.

Particularly, according to the test examples of the present invention, the cosmetic composition (Example 1) for a leave-on type mask sheet according to the present invention was determined for skin irritation in comparison with a cosmetic composition (Comparative Example 1) including a chemical surfactant for a cleansing type mask sheet. After the test, it was shown that the cosmetic composition (Example 1) for a mask sheet according to the present invention causes little skin irritation and pruritus, but the cosmetic composition (Comparative Example 1) including a chemical surfactant for a mask sheet causes more irritation and pruritus as compared to the cosmetic composition according to the present invention. It is thought that Comparative Example 1 includes a chemical surfactant, and thus causes irritation and pruritus when it is not washed off. However, the cosmetic composition according to the present invention includes saponin instead of a chemical surfactant, and thus causes little skin irritation. As can be seen from the results, the cosmetic composition for a mask sheet according to the present invention can be applied for a leave-on type mask sheet even though it has a cleansing effect (see, Test Example 2).

In addition, the cosmetic composition for a mask sheet according to the present invention has skin wrinkle-improving and whitening effects.

Particularly, according to the test examples of the present invention, the cosmetic composition (Examples 2 and 3) for a leave-on type mask sheet according to the present invention was determined for skin wrinkle-improving and whitening effects in comparison with a cosmetic composition (Comparative Examples 2 and 3) including a chemical surfactant for a cleansing type mask sheet. After the test, in the case of Examples 2 and 3 according to the present invention, many subjects felt skin wrinkle-improving and whitening effects significantly. However, in the case of Comparative Examples 2 and 3, many subjects felt no skin wrinkle-improving and whitening effects. It is thought that Examples 2 and 3 according to the present invention are leave-on types and functional ingredients thereof are not washed off with water, and the active ingredients of saponin realize their effects, thereby providing a synergic effect in terms of wrinkle-improving and skin whitening, unlike Comparative Examples. Therefore, it can be seen that the cosmetic composition for a mask sheet according to the present invention can be used for skin wrinkle-improving and skin whitening applications as well as cleansing and moisturizing applications (see, Test Examples 3 and 4).

According to the present invention, the cosmetic composition may include 0.01-40 wt % of a fluorinated compound, 0.01-40 wt % of saponin, 1-40 wt % of a moisturizing agent, 0.01-10 wt % of a gelling agent and 55-90 wt % of purified water. Preferably, the cosmetic composition may include 0.01-20 wt % of a fluorinated compound, 0.01-20 wt % of saponin, 1-20 wt % of a moisturizing agent, 0.01-5 wt % of a gelling agent and 55-90 wt % of purified water. In addition, the cosmetic composition may further include a metal ion chelating agent and a pH modifying agent. Preferably, the cosmetic composition may further include 0.01-2.0 wt % of a metal ion chelating agent and 0.01-4.0 wt % of a pH modifying agent based on the total weight of the cosmetic composition.

According to the present invention, the fluorinated compound is at least one selected from the group consisting of methyl perfluorobutyl ether, methyl perfluoroisobutyl ether, ethyl perfluorobutyl ether, ethyl perflruoroisobutyl ether and isopentane.

The fluorinated compound is a colorless, odorless, low-irritating and non-volatile organic compound, has ability as oxygen carrier, shows high vaporizability and has a cleaning effect to light oil. According to the present invention, the fluorinated compound is used preferably in an amount of 0.01-40 wt % based on the total weight of the composition. When the content of fluorinated compound is less than 0.01 wt %, it is not possible to provide a sufficient foaming effect to be accomplished by the present invention. When the content of fluorinated compound is larger than 40 wt %, the fluorinated compound has increased vaporizability to cause degradation of utility undesirably.

Saponin is a general term of triterpene and steroid-based glycosides distributed widely in the vegetable kingdom and functions as a naturally occurring surfactant generating foam continuously when it is shaken with water. According to the present invention, saponin is not particularly limited, as long as it is extracted from ginseng, Sapindus mukorossi GAERTNER, Quillaja, soybean, green tea, potato, bellflower root, cabbage, carrot, parsley, Chinese Liquorice, ginger and garlic.

Extraction of saponin may be carried out by the methods known to those skilled in the art. For example, shells of Sapindus mukorossi GAERTNER fruits are dipped in water to extract the ingredients in the shells, and then filtered through a filter to obtain pure extract of Sapindus mukorossi GAERTNER fruits. Then, the extract is dried to remove water, thereby providing powdery saponin.

According to the present invention, saponin is used preferably in an amount of 0.01-40 wt % based on the total weight of the composition. When the content of saponin is less than 0.01%, it is not possible to generate foam sufficiently, when saponin meets with the fluorinated compound. When the content of saponin is larger than 40 wt %, foam is generated excessively, thereby making it difficult to use the cosmetic composition for a leave-on type mask sheet.

According to the present invention, the moisturizing agent is at least one selected from the group consisting of 1,3-butylene glycol, glycerin, dipropylene glycol, isopentyl glycol, propanediol, 1,2-hexanediol, caprylyl glycol, ethylhexyl glycerin, polyglycerin-3, betain and sorbitol. According to the present invention, the moisturizing agent is used preferably in an amount of 1-20 wt % based on the total weight of the composition. When the content of moisturizing agent is not within the above-defined range, it is not possible to provide the skin with a moisturizing effect.

According to the present invention, the gelling agent is at least one selected from the group consisting of acrylic gelling agents, such as acrylate copolymers, acrylate/C10-30 alkyl acrylate crosspolymers or sodium acrylate, gum-like gelling agents, such as xanthan gum or cellulose gum, and carbomers. According to the present invention, the gelling agent is used preferably in an amount of 0.01-5.0 wt % based on the total weight of the composition. When the content of gelling agent is less than 0.01 wt %, it is not possible to obtain an effect of increasing viscosity sufficiently. When the content of gelling agent is larger than 5.0 wt %, viscosity is increased excessively, and thus the composition cannot infiltrate to a mask sheet, when the mask sheet is impregnated with the composition, and absorbability is degraded undesirably, when applying the composition to the skin.

In addition, the metal ion chelating agent that may be further incorporated to the cosmetic composition according to the present invention is at least one selected from the group consisting of ethylene diamine tetraacetic acid (EDTA), disodium EDTA, phosphoric acid, citric acid, ascorbic acid, succinic acid, gluconic acid, sodium polyphosphate and sodium metaphosphate. The metal ion chelating agent may be used preferably in an amount of 0.1-2.0 wt % based on the total weight of the composition. When the content of metal ion chelating agent is not within the above-defined range, it is not possible to provide a function as a metal ion chelating agent sufficiently or skin irritation may occur.

The pH modifying agent that may be further incorporated to the cosmetic composition according to the present invention is at least one selected from the group consisting of citric acid, sodium citrate, alginine, potassium hydroxide, triethanolamine, tromethamine, sodium hydroxide and potassium hydroxide. The pH modifying agent may be used preferably in an amount of 0.01-4.0 wt % based on the total weight of the composition. When the content of pH modifying agent is not within the above-defined range, it is not possible to satisfy a pH of 3.0-9.0, the standard defined by the cosmetic legislation. In this case, the cosmetic composition cannot be used for leave-on application.

According to the present invention, the mask sheet is made of cotton, a non-woven web, rayon-containing material, cellulose material or a hydrogel material.

Hereinafter, the method for preparing the cosmetic composition for a leave-on type mask sheet according to the present invention will be explained in detail.

First, an aqueous portion including a metal ion chelating agent, pH modifying agent, moisturizing agent and purified water is dissolved completely and a gelling agent is added thereto. Next, a fluorinated compound (volatile material) is introduced to the resultant mixture while carrying out agitation at 2000 rpm with a homo-mixer to obtain emulsion. Then, saponin is added to the resultant emulsion under agitation to obtain a gel-state or liquid composition. The composition and a mask sheet are introduced to a pouch and the pouch is sealed to obtain a cosmetic composition for a leave-on type mask sheet.

### Advantageous Effects

As described above, the cosmetic composition for a leave-on type mask sheet according to the present invention includes saponin instead of a chemical surfactant, and thus can reduce side effects, such as skin irritation, alleviate inconvenience of washing-off after using the mask sheet, increase a moisturizing effect by virtue of elimination of such washing-off procedure, and can provide a cleansing effect, such as an effect of removing sebum or waste by foam, through a self-foaming function. In addition, it is possible to prevent loss of the functional ingredients contained in the cosmetic composition and wrinkle-improving and skin-whitening active ingredients of saponin during washing, and thus can provide higher wrinkle-improving and skin-whitening effects. Further, the cosmetic composition also shows a cleansing effect of removing sebum and waste by using foam generated by self-foaming of saponin and the fluorinated compound.

### BRIEF DESCRIPTION OF DRAWINGS

FIG. 1 is a photographic view illustrating actual use of the composition according to an embodiment of the present invention, after a mask sheet is impregnated with the composition.

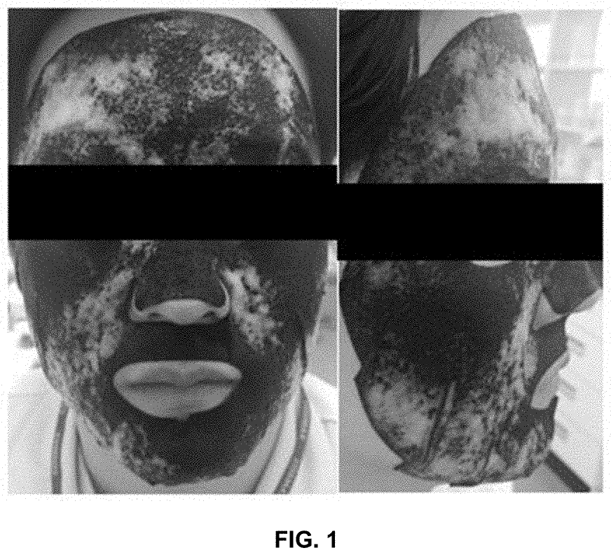

FIG. 2 is a photographic view illustrating the composition according to an embodiment of the present invention.

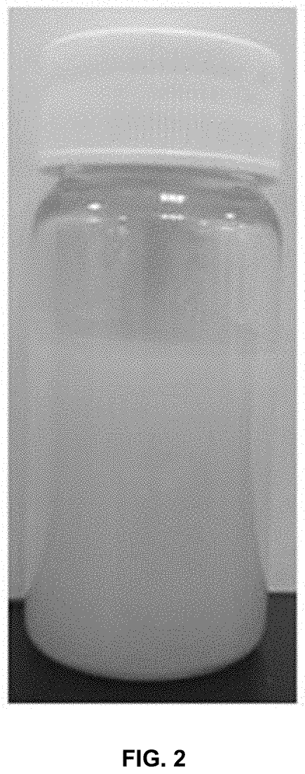

### DETAILED DESCRIPTION

Exemplary embodiments now will be described more fully hereinafter. The present invention may, however, be embodied in many different forms and should not be construed as limited to the exemplary embodiments set forth therein.

### Example 1: Preparation of Cosmetic Composition for Leave-on Type Mask Sheet

A cosmetic composition was prepared according to the composition as shown in the following Table 1.

Disodium EDTA, glycerin and a preservative were added to purified water and agitated/dissolved therein. After checking complete dissolution, polyacrylamide was introduced while carrying out agitation. Agitation was carried out for 10 minutes until polyacrylamide was dissolved completely. After checking that polyacrylamide was dissolved without aggregation, a fragrance was introduced thereto and agitated therein. After carrying out agitation for 3 minutes, the resultant mixture was cooled to 25° C. Herein, it is essentially required to check that the temperature was decreased to 25° C. Then, air bubbles in the content were removed through a deaeration process. After that, ethyl perfluorobutyl ether and ethyl perfluoroisobutyl ether were introduced while carrying out agitation. After checking that emulsion was formed, saponin dissolved in a separate container was added to the emulsion and the resultant emulsion was agitated thoroughly for 3 minutes. Herein, agitation was carried out merely by using paddles, not a homo-mixer.

Herein, saponin was obtained by dipping shells of Sapindus mukorossi GAERTNER fruits in water to extract the materials in the shells and carrying out filtering through a filter to obtain extract of Sapindus mukorossi GAERTNER fruits. Then, the extract was dried to remove purified water, thereby providing powdery saponin.

After that, acrylate copolymer dissolved in a separate container was introduced and agitation was carried out for 3 minutes. After carrying out agitation sufficiently, a neutralizing agent was introduced and agitation was carried out sufficiently for 3 minutes.

When a non-woven web was impregnated with the composition obtained according to the composition as shown in Table 1, foam was generated.

TABLE 1
Example 1

| No. | Ingredients | Amount (wt %) |
|---|---|---|
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Fragrance | 0.05 |
| 7 | Ethyl perfluorobutyl ether, Ethyl | 5 |
| | perfluoroisobutyl ether | |
| 8 | Saponin | 1 |
| 9 | Acrylate copolymer | 2 |
| 10 | Triethanolamine | 0.2 |

In Table 1, Ingredient 2 is a metal ion chelating agent, Ingredients 3 and 4 are moisturizing agents, Ingredient 5 is an emulsifying agent/gelling agent, Ingredient 7 is a fluorinated compound, Ingredient 9 is a gelling agent, and Ingredient 10 is a neutralizing agent.

### Comparative Example 1: Preparation of Cosmetic Composition Including Chemical Surfactant for Cleansing Type Mask Sheet

The following Table 2 shows the ingredients contained in a cosmetic composition including a chemical surfactant for a cleansing type mask sheet. The composition was prepared as follows.

Disodium EDTA, glycerin and a preservative were added to purified water and agitated/dissolved therein. After checking complete dissolution, polyacrylamide was introduced while carrying out agitation. Agitation was carried out for 10 minutes until polyacrylamide was dissolved completely. After checking that polyacrylamide was dissolved without aggregation, a fragrance was introduced thereto and agitated therein. After carrying out agitation for 3 minutes, the resultant mixture was cooled to 25° C. Herein, it is essentially required to check that the temperature was decreased to 25° C. Then, air bubbles in the content were removed through a deaeration process. After that, ethyl perfluorobutyl ether and ethyl perfluoroisobutyl ether were introduced while carrying out agitation. After checking that emulsion was formed, preliminarily weighed sodium cocoyl apple aminoacid and potassium cocoyl glycinate were introduced. Herein, agitation was carried out merely by using paddles, not a homo-mixer. After that, acrylate copolymer dissolved in a separate container was introduced and agitation was carried out for 3 minutes. After carrying out agitation sufficiently, a neutralizing agent was introduced and agitation was carried out sufficiently for 3 minutes.

When a non-woven web was impregnated with the composition obtained according to the composition as shown in Table 2, foam was generated. After foam was generated sufficiently, it should be washed off.

TABLE 2

| | Comparative Example 1 | |
|---|---|---|
| No. | Ingredients | Amount (wt %) |
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Ethyl perfluorobutyl ether, Ethyl | 5 |
| | perfluoroisobutyl ether | |
| 7 | Sodium cocoyl apple aminoacid | 3 |
| 8 | Potassium cocoyl glycinate | 5 |
| 9 | Acrylate copolymer | 2 |
| 10 | Triethanolamine | 0.2 |

In Table 2, Ingredient 2 is a metal ion chelating agent, Ingredients 3 and 4 are moisturizing agents, Ingredient 5 is an emulsifying agent/gelling agent, Ingredient 6 is a fluorinated compound, Ingredients 7 and 8 are chemical surfactants, Ingredient 9 is a gelling agent, and Ingredient 10 is a neutralizing agent.

### Test Example 1: Functional Test for Cleansing, Moisturizing and Emollient Effects

Example 1 and Comparative Example 1 were prepared and used for a test for twenty (20) healthy male/female subjects.

The test was carried out by allowing the subjects to use each of the non-woven webs impregnated with Example 1 and Comparative Example 1 for 15 minutes and judging feelings of use, such as sebum improvement and moisturizing and skin emollient effects. The results are shown in the following Table 3 (Example 1) and Table 4 (Comparative Example 1). The non-woven webs used for the test were made of a cupra material. Evaluation was carried out according to the following criteria.

5: Excellent, 4: good, 3: satisfactory, 2: unsatisfactory, 1: poor.

TABLE 3

| Male | A | B | C | D | E | F | G | H | I | J |
|---|---|---|---|---|---|---|---|---|---|---|
| Sebum | 4 | 5 | 4 | 4 | 5 | 5 | 4 | 4 | 5 | 4 |
| improvement | | | | | | | | | | |
| Moisturizing | 5 | 4 | 5 | 5 | 4 | 4 | 5 | 4 | 5 | 5 |
| effect | | | | | | | | | | |
| Emollient | 4 | 5 | 4 | 3 | 5 | 4 | 5 | 3 | 4 | 4 |
| effect | | | | | | | | | | |
| Female | K | L | M | N | O | P | Q | R | S | T |
| Sebum | 5 | 4 | 4 | 3 | 4 | 5 | 4 | 4 | 3 | 4 |
| improvement | | | | | | | | | | |
| Moisturizing | 4 | 5 | 5 | 5 | 4 | 5 | 5 | 5 | 4 | 5 |
| effect | | | | | | | | | | |
| Emollient | 5 | 5 | 5 | 4 | 4 | 3 | 5 | 5 | 5 | 4 |
| effect | | | | | | | | | | |

TABLE 4

| Male | A | B | C | D | E | F | G | H | I | J |
|---|---|---|---|---|---|---|---|---|---|---|
| Sebum | 4 | 5 | 5 | 4 | 5 | 5 | 4 | 4 | 4 | 4 |
| improvement | | | | | | | | | | |
| Moisturizing | 3 | 2 | 4 | 3 | 3 | 2 | 3 | 2 | 5 | 3 |
| effect | | | | | | | | | | |
| Emollient | 2 | 2 | 2 | 3 | 3 | 3 | 2 | 3 | 2 | 2 |
| effect | | | | | | | | | | |
| Female | K | L | M | N | O | P | Q | R | S | T |
| Sebum | 4 | 4 | 4 | 4 | 4 | 5 | 4 | 3 | 5 | 4 |
| improvement | | | | | | | | | | |
| Moisturizing | 2 | 2 | 3 | 3 | 3 | 4 | 2 | 2 | 3 | 4 |
| effect | | | | | | | | | | |
| Emollient | 2 | 3 | 3 | 3 | 2 | 4 | 2 | 2 | 3 | 3 |
| effect | | | | | | | | | | |

The functional test results from the overall comparison of Example 1 (Table 3) with Comparative Example 1 (Table 4) are shown in the following Table 5.

TABLE 5

| | | Comparative |
|---|---|---|
| | Example 1 | Example 1 |
| Male | | |
| Sebum improvement | 4.4 | 4.4 |
| Moisturizing effect | 4.6 | 3 |
| Emollient effect | 4.1 | 2.4 |
| Female | | |
| Sebum improvement | 4 | 4.1 |
| Moisturizing effect | 4.7 | 2.8 |
| Emollient effect | 4.5 | 2.7 |

As can be seen from the results, in both male and female subjects, Example 1 provides improved moisturizing and skin emollient effects, which are insufficient in the case of Comparative Example 1 according to the related art, while maintaining a sebum-improving effect (skin cleansing effect), which is a unique function of a self-foaming mask sheet.

### Test Example 2: Skin Irritation Test

Example 1 and Comparative Example 1 were prepared and used for a test for ten (10) healthy male/female subjects.

The test was carried out by allowing the subjects to use each of the non-woven webs impregnated with Example 1 and Comparative Example 1 for 15 minutes and judging irritation and pruritus. The results are shown in the following Table 6. The non-woven webs used for the test were made of a cupra material. Evaluation was carried out according to the following criteria.

4: Strong irritation, 3: medium irritation, 2: light irritation, 1: little irritation, 0: no irritation.

TABLE 6

| Example 1 | A | B | C | D | E | F | G | H | I | J | Average |
|---|---|---|---|---|---|---|---|---|---|---|---|
| Irritation | 0 | 0 | 0 | 0 | 0 | 1 | 0 | 0 | 0 | 1 | 0.2 |
| Pruritus | 1 | 1 | 1 | 0 | 0 | 1 | 0 | 1 | 1 | 1 | 0.7 |
| Comp. Ex. 1 | A | B | C | D | E | F | G | H | I | J | Average |
| Irritation | 2 | 3 | 2 | 3 | 2 | 3 | 3 | 2 | 4 | 4 | 2.8 |
| Pruritus | 3 | 3 | 2 | 3 | 1 | 2 | 4 | 2 | 4 | 3 | 2.7 |

As can be seen from the results of Table 6, Example 1 according to the present invention causes little irritation and pruritus, while Comparative Example 1 causes more irritation and pruritus as compared to the present invention. It is thought that Comparative Example 1 includes a chemical surfactant, and thus the chemical surfactant remaining on the skin causes irritation and pruritus when it is not washed off.

### Test Example 3: Skin Wrinkle-Improving Test

The mask sheets according to Example 2 (Table 7) and Comparative Example 2 (Table 8) were used to carry out a panel test for skin wrinkle-improving effects.

First, thirty (30) healthy females in their 35-50 ages were divided into two groups each of which had 15 females. The first group and the second group were allowed to apply the mask sheet according to Example 2 and the mask sheet according to Comparative Example 2, respectively, to the face portion once every other day for two months. After two months, a degree of improvement in skin wrinkles was evaluated through the survey of subjects. With reference to skin wrinkle improvement and elasticity enhancement, evaluation was performed in three levels of no improvement, slight improvement and significant improvement. The results are shown in the following Table 7.

TABLE 7
Example 2

| No. | Ingredients | Amount (wt %) |
|---|---|---|
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Fragrance | 0.05 |
| 7 | Ethyl perfluorobutyl ether, Ethyl | 5 |
| | perfluoroisobutyl ether | |
| 8 | Saponin | 1 |
| 9 | Acrylate copolymer | 2 |
| 10 | Triethanolamine | 0.2 |
| 11 | Adenosine | 0.04 |

In Table 7, Ingredient 2 is a metal ion chelating agent, Ingredients 3 and 4 are moisturizing agents, Ingredient 5 is an emulsifying agent/gelling agent, Ingredient 7 is a fluorinated compound, Ingredient 9 is a gelling agent, ingredient 10 is a neutralizing agent, and Ingredient 11 is a wrinkle-improving agent.

TABLE 8
Comparative Example 2

| No. | Ingredients | Amount (wt %) |
|---|---|---|
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Ethyl perfluorobutyl ether, Ethyl | 5 |
| | perfluoroisobutyl ether | |
| 7 | Sodium cocoyl apple aminoacid | 3 |
| 8 | Potassium cocoyl glycinate | 5 |
| 9 | Acrylate copolymer | 2 |
| 10 | Triethanolamine | 0.2 |
| 11 | Adenosine | 0.04 |

In Table 8, Ingredient 2 is a metal ion chelating agent, Ingredients 3 and 4 are moisturizing agents, Ingredient 5 is an emulsifying agent/gelling agent, Ingredient 6 is a fluorinated compound, Ingredients 7 and 8 are chemical surfactants, Ingredient 9 is a gelling agent, Ingredient 10 is a neutralizing agent, and Ingredient 11 is a wrinkle-improving agent.

TABLE 9

| | No | Slight | Significant |
|---|---|---|---|
| | improvement | improvement | improvement |
| Example 2 | 1 | 4 | 10 |
| Comparative | 13 | 2 | 0 |
| Example 2 | | | |

As can be seen from the results of Table 9, many subjects felt a significantly improved effect in the case of Example 2 according to the present invention. On the contrary, in the case of Comparative Example 2, more subjects felt no skin wrinkle improvement.

### Test Example 4: Skin Whitening Effect Test

The mask sheets according to Example 3 (Table 10) and Comparative Example 3 (Table 11) were used to carry out a panel test for skin whitening effects.

First, thirty (30) healthy females in their 35-50 ages were divided into two groups each of which had 15 females. The first group and the second group were allowed to apply a portion of the mask sheet according to Example 3 and a portion of the mask sheet according to Comparative Example 3, respectively, to the upper arm once a day for one month. Before the test, pigmentation was made in each subject by using a UV irradiator. The whitening effect was evaluated by observing the skin color by the naked eyes. The results are shown in the following Table 12.

TABLE 10

| | Example 3 | |
|---|---|---|
| No. | Ingredients | Amount (wt %) |
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Fragrance | 0.05 |
| 7 | Ethyl perfluorobutyl ether, Ethyl | 5 |
| | perfluoroisobutyl ether | |
| 8 | Saponin | 1 |
| 9 | Acrylate copolymer | 2 |
| 10 | Triethanolamine | 0.2 |
| 11 | Niacin amide | 2.0 |

In Table 10, Ingredient 2 is a metal ion chelating agent, Ingredients 3 and 4 are moisturizing agents, Ingredient 5 is an emulsifying agent/gelling agent, Ingredient 7 is a fluorinated compound, Ingredient 9 is a gelling agent, ingredient 10 is a neutralizing agent, and Ingredient 11 is a skin whitening functional agent.

TABLE 11
Comparative Example 3

| No. | Ingredients | Amount (wt %) |
|---|---|---|
| 1 | Purified water | to 100 |
| 2 | Disodium EDTA | 0.02 |
| 3 | Glycerin | 5 |
| 4 | 1,2-hexanediol/ethylhexyl glycerin | 2.0/0.04 |
| 5 | Polyacrylamide | 1 |
| 6 | Ethyl perfluorobutyl ether, Ethyl | 5 |
| | perfluoroisobutyl ether | |
| 7 | Sodium cocoyl apple aminoacid | 3 |
| 8 | Potassium cocoyl glycinate | 5 |
| 9 | Acrylate copolymer | 2 |
| 10 | Triethanolamine | 0.2 |
| 11 | Niacin amide | 2.0 |

In Table 11, Ingredient 2 is a metal ion chelating agent, Ingredients 3 and 4 are moisturizing agents, Ingredient 5 is an emulsifying agent/gelling agent, Ingredient 6 is a fluorinated compound, Ingredients 7 and 8 are chemical surfactants, Ingredient 9 is a gelling agent, Ingredient 10 is a neutralizing agent, and Ingredient 11 is a skin whitening functional agent.

TABLE 12

| | No | Slight | Significant |
|---|---|---|---|
| | improvement | improvement | improvement |
| Example 3 | 1 | 5 | 9 |
| Comparative | 11 | 3 | 0 |
| Example 3 | | | |

As can be seen from the results of Table 12, many subjects felt a significantly improved effect in the case of Example 3 according to the present invention. On the contrary, in the case of Comparative Example 3, more subjects felt no skin wrinkle improvement.

### INDUSTRIAL APPLICABILITY

As described hereinabove, the cosmetic composition for a leave-on type mask sheet according to the present invention includes saponin instead of a chemical surfactant, and thus can reduce side effects, such as skin irritation, alleviate inconvenience of washing-off after using the mask sheet, improve a moisturizing effect by virtue of elimination of the washing-off procedure, and provide a cleansing effect, such as an effect of removing skin sebum or waste and wrinkle-improving and skin-whitening effects.

What is claimed is:

1. A cosmetic composition for a leave-on type mask sheet comprising a fluorinated compound and saponin as a self-foaming agent.

2. The cosmetic composition for a leave-on type mask sheet according to claim 1, which comprises no chemical surfactant.

3. The cosmetic composition for a leave-on type mask sheet according to claim 1, which is for combined use of cleansing, moisturizing, wrinkle-improving and whitening.

4. The cosmetic composition for a leave-on type mask sheet according to claim 1, which comprises 0.01-20 wt % of a fluorinated compound, 0.01-20 wt % of saponin, 1-20 wt % of a moisturizing agent, 0.01-5 wt % of a gelling agent and 55-90 wt % of purified water.

5. The cosmetic composition for a leave-on type mask sheet according to claim 1, wherein the fluorinated compound is at least one selected from the group consisting of methyl perfluorobutyl ether, methyl perfluoroisobutyl ether, ethyl perfluorobutyl ether, ethyl perflruoroisobutyl ether and isopentane.

6. The cosmetic composition for a leave-on type mask sheet according to claim 4, wherein the moisturizing agent is at least one selected from the group consisting of 1,3-butylene glycol, glycerin, dipropylene glycol, isopentyl glycol, propanediol, 1,2-hexanediol, caprylyl glycol, ethylhexyl glycerin, polyglycerin-3, betain and sorbitol.

7. The cosmetic composition for a leave-on type mask sheet according to claim 4, wherein the gelling agent is at least one selected from the group consisting of acrylic gelling agents, such as acrylate copolymers, acrylate/C10-30 alkyl acrylate crosspolymers or sodium acrylate, gum-like gelling agents, such as xanthan gum or cellulose gum, and carbomers.

8. The cosmetic composition for a leave-on type mask sheet according to claim 1, wherein the mask sheet is made of cotton, a non-woven web, rayon-containing material, cellulose material or a hydrogel material.
